(19) United States
(12) Patent Application Publication
(10) Pub. No.: US 2022/0409633 A1
(43) Pub. Date: Dec. 29, 2022

(54) COMPOSITION COMPRISING BUDESONIDE FOR OPHTHALMIC USE

(71) Applicant: NTC S.R.L., Milano (MI) (IT)

(72) Inventors: Luciano MARCELLONI, Milano (MI) (IT); Federico BERTOCCHI, Milano (MI) (IT)

(21) Appl. No.: 17/777,964

(22) PCT Filed: Dec. 3, 2020
(86) PCT No.: PCT/IB2020/061444

§ 371 Date: May 18, 2022

(30) Foreign Application Priority Data
Dec. 3, 2019 (IT) 102019000022860
Dec. 3, 2019 (IT) 102019000022881

(51) Int. Cl. A61K 31/58; A61P 29/00; A61K 9/16

(52) U.S. Cl. CPC A61K 31/58; A61P 29/00; A61K 9/1623

(57) ABSTRACT

The present invention relates to budesonide, or a salt thereof, or mixtures thereof for ophthalmic use in a method for the curative treatment of an eye disorder or ailment or disease in a subject in need. Further, the present invention relates to a composition comprising a mixture comprising or, alternatively, consisting of: (i) a budesonide, or a salt thereof, or mixtures thereof; (ii) at least one suspending/thickening agent, and optionally one or more physiologically and/or pharmacologically acceptable excipients; said composition being for use in a method for the curative treatment of an inflammation of the and ocular adnexa or inflammation of the eyeball in a subject in need.

The present invention relates to budesonide, or a salt thereof, or mixtures thereof for ophthalmic use in a method for the curative treatment of an eye disorder or ailment or disease in a subject in need, preferably wherein said method comprises an administration of budesonide to said subject through the ocular topical route.

Furthermore, the present invention relates to a composition comprising a mixture comprising or, alternatively, consisting of: (i) a budesonide, or a salt thereof, or mixtures thereof; (ii) at least one suspending/thickening agent, and optionally (iii) one or more physiologically and/or pharmacologically acceptable excipients; said composition being for use in a method for the curative treatment of an inflammation of the ocular adnexa or inflammation of the eyeball in a subject in need. Furthermore, forming an object of the present invention is a method for the curative treatment of an inflammation of the ocular adnexa or inflammation of the eyeball in a subject in need, wherein said treatment method comprises administering said composition to said subject.

The ocular adnexa include the ocular muscles, the eyelids, the lacrimal apparatus. Numerous anatomical structures provide the eye with protection, lubrication of the surface exposed to the external, possibility of movement. At the front, the eyelids constitute curtains, which, if necessary, close the orbital cavity. The lacrimal apparatus, in its secretion portion (main and accessory lacrimal glands), and outflow (canals, lacrimal sac, nose-lacrimal duct), provides for the lubrication and hydration of exposed surfaces and the removal of dirt. The conjunctiva forms sacs that allow ocular movements, protecting the sclera and closing the orbital cavity. The 6 extrinsic eye muscles, inserted on the orbital walls and on the sclera, move the eye.

The eye, or eyeball, is the main sense organ of the visual apparatus, which has the task of obtaining information about the surrounding environment through light. The conjunctiva is the membrane that internally covers the eyelid and folds to cover the sclera (the robust white fibrous membrane that covers the eye) up to the edge of the cornea (the transparent layer in front of iris and pupil). The conjunctiva protects the eye by keeping foreign bodies and microorganisms that cause infections away and contributing to the maintenance of the tear film. The most frequent condition of the conjunctiva is inflammation (conjunctivitis). There are many causes of inflammation, including bacterial infections (including chlamydia), viruses or fungi; allergic reactions; chemical substances or foreign bodies in the eye; and excessive exposure to sunlight. The cornea is the transparent part of the eye found in front of the pupil and the iris (the coloured part of the eye).

Today, numerous pharmaceutical products are available for the treatment of inflammatory diseases of the eye, useful to reduce inflammation that may affect the ocular adnexa or the eyeball. Said pharmaceutical products for the treatment of inflammatory diseases contain non-steroidal anti-inflammatory active substances (NSAIDs; non-steroidal anti-inflammatory drugs), or steroidal (corticosteroids). Non-steroidal active substances act on the metabolism of arachidonic acid and eicosapentaenoic acid, precursors of molecules involved in the inflammatory process such as prostaglandins (PG), prostacyclins (PC), thromboxanes (TX) and leukotrienes (LT).

Steroidal active substances possess a sterol nucleus (cyclopentanoperhydrophenanthrene) consisting of four fused rings—three six-atom rings (A, B, C) and a five-atom ring (D)—as represented in the following general formula (I):

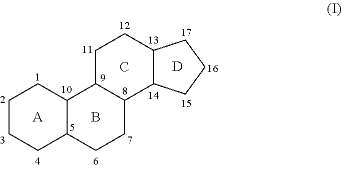

The molecule called budesonide (CAS No. 51333-22-3) is a steroidal anti-inflammatory agent used locally in some topical regions, being used in particular through the inhalation route in the prevention of asthma attacks (in the form of a suspension by nebulisation for inhalations or nasal spray, or in the form of dry powder for inhalation), or through the enteral route in the treatment of Crohn's disease (in the form of a coated capsule). When budesonide reaches the liver carried by the bloodstream, budesonide is metabolised to pharmacologically inactive by-products. For this reason, budesonide is used only locally. Budesonide has peculiarities of action that make it an active ingredient with an “inherently delayed” action. In (Magnus JENDBRO et al. “Pharmacokinetics of budesonide and its major ester metabolite after inhalation and (ravenous administration of budesonide in the ran”; Drug Metabolism and Disposition, The American Society of Pharmacology and Experimental Therapeutics, Vol. 29, No. 5 (1998); Elisabet WIESLANDER et al. “Pharmacologic Importance of the Reversible Fatty Acid Anjugation of Budesonide Studied in a Rat Cell Line In Vitro”; American Journal of Respiratory Cell and Molecular Biology; Vol 19 (1998), 477-484″) it has been shown that such delayed action is due to a longer action time with respect to other corticosteroids (for example loteprednol or dexamethasone), necessary for reversible intracellular esterification of budesonide to occur, in an esterified form and in a non-esterified form.

Budesonide has the following general formula (ID:

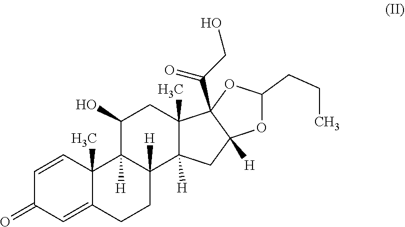

Budesonide is not water-soluble. The pharmaceutical formulations based on budesonide, present on the market, therefore provide for the presence of said steroidal anti-inflammatory agent in solid form (for example as powder, granulate or tablets), or in aqueous suspension.

Budesonide aqueous suspension formulations are recommended for the local symptomatic treatment of allergic rhinitis. Such symptoms comprise swelling of the mucous membranes due to an inflammation and phlogosis originating from pollen, dust, mites or other foreign antigenic materials. Said aqueous suspension formulations used in the nasal region help to successfully limit the inflammations induced by allergic reactions.

The suspension budesonide formulations theoretically further prolong the inherently delayed action of budesonide, by virtue of a slow process of dissolution of such corticosteroid, which is therefore not immediately available for absorption since they are not dissolved in the biological fluid outside the tissue.

There are several eye drops based on non-steroidal anti-inflammatories (NSAIDs) on the market. The main drawbacks of NSAID-based eye drops are conjunctival redness and burning, eyelid oedema and anterior segment structures of the eye, keratitis and itching. Steroid eye drops containing loteprednol (Lotemax®) or dexamethasone (Maxidex®) also have side effects including: feeling of discomfort in the eye, feeling of foreign body in the eye, corneal defects, ocular itching, increased lacrimation, irritation, redness.

The prior document CN 101 987 101 A relates to an anti-inflammatory ophthalmic composition comprising a glucocorticoid, preferably budesonide, cited in a first list comprising tens of possible alternatives. Said composition is preferably eye drops in the form of an ophthalmic solution or suspension, preferably an ophthalmic ointment or an ophthalmic gel. The ophthalmic gel comprises one or more from a buffer agent, an isotonic agent, an antibacterial agent, a stabiliser agent, an antioxidant, a surfactant, an acidity-basicity regulator, a chelating agent, an absorption enhancer, a thickening agent, a wetting agent (second list of components). A stabiliser agent for said gel is carboxymethylcellulose, selected from a third list comprising a series of possible alternatives.

None of the compositions discussed in the examples of prior document CN 101 987 101 A contains budesonide, nor only the soluble fractions of carboxymethylcellulose. Furthermore, in such examples, budesonide in the form of micronized powder is not present.

Prior document US 2009/312724 A1 discloses aqueous solution corticosteroid formulations for use in the treatment of nasal and/or ophthalmic diseases or symptoms. In an embodiment the corticosteroid is budesonide. Paragraph [0152] of prior document US 2009/312724 A1 specifies that, in order to maximise nasal administration, said formulation may have a mass median aerodynamic diameter (MMAD) of at least 3.5 μm, of at least 5 μm, of at least 10 μm, of at least 20 μm or of at least 35 μm. Paragraph [0179] of such document discloses commercial formulations of budesonide. Among these, PULMICORT RESPULES suspension, is a sterile aqueous suspension of micronized budesonide which can be administered by inhalation by means of a nebuliser.

None of the formulations discussed in the examples of prior document US 2009/312724 A1 contains only the soluble fractions of sodium carboxymethylcellulose. Furthermore, none of the formulations shown in such document uses micronized budesonide for use in a method for the curative treatment of an inflammation of the ocular adnexa or inflammation of the eyeball.

The need is therefore felt to be able to have a composition capable of allowing to obtain an anti-inflammatory action that is effective and prolonged over time so as to avoid multiple administrations which unnecessarily increase the feeling of burning and itching. Furthermore, the need is felt to be able to have a composition which is not affected by the existing problems and limits of the currently existing formulations, and which is tolerated by a greater number of subjects.

After a long and intense research and development activity, the Applicant developed an ophthalmic composition capable of providing an adequate response to the limits, drawbacks and problems currently existing in products on the market.

Thus, forming an object of the present invention is budesonide, or a salt thereof, or mixtures thereof for ophthalmic use in a method for the curative treatment of an eye disorder or ailment or disease in a subject in need, preferably wherein said method comprises an administration of said budesonide to said subject through the ocular topical route, having the characteristics as defined in the attached claims.

Furthermore, forming an object of the present invention is a composition comprising a mixture comprising or, alternatively, consisting of: (i) a budesonide, or a salt thereof, or mixtures thereof; (ii) at least one suspending/thickening agent, and optionally (iii) one or more physiologically and/or pharmacologically acceptable excipients, wherein said composition is for use in a method for the curative treatment of an inflammation of the ocular adnexa or of the eyeball in a subject in need, having the characteristics as defined in the attached claims.

Furthermore, forming an object of the present invention is a method for the curative treatment of inflammation of the ocular adnexa or an inflammation of the eyeball in a subject in need, wherein said treatment method comprises administering said composition to said subject, having the characteristics as defined in the attached claims.

Preferred embodiments of the present invention will be described hereinafter by way of non-limiting example with the aid of the attached drawings, wherein some indices of the eye inflammatory condition are measured:

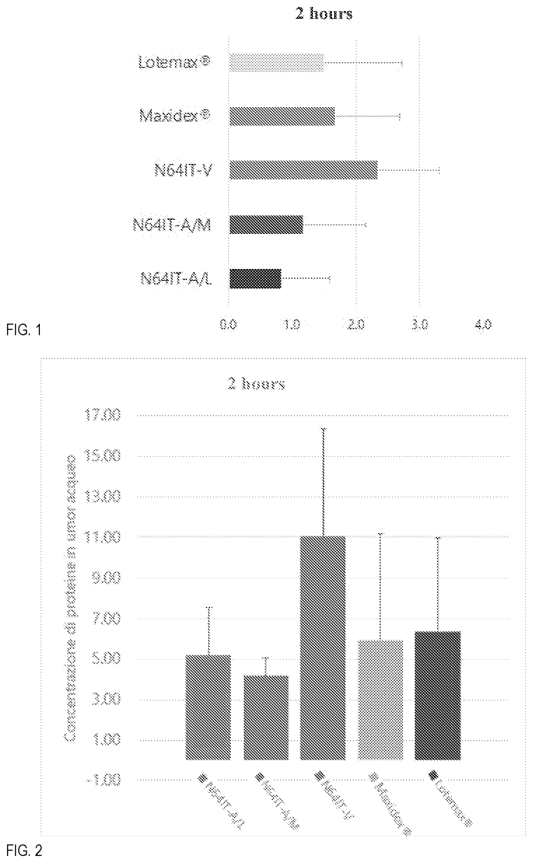

FIG. 1 shows—in graphic form—the results of the first observations of Example 2 relating to the possible material suspended in water after 2 hours;

FIG. 2 shows—in graphic form the results of the second observations of Example 2 relating to the protein concentration (as an index of inflammation) after 2 hours;

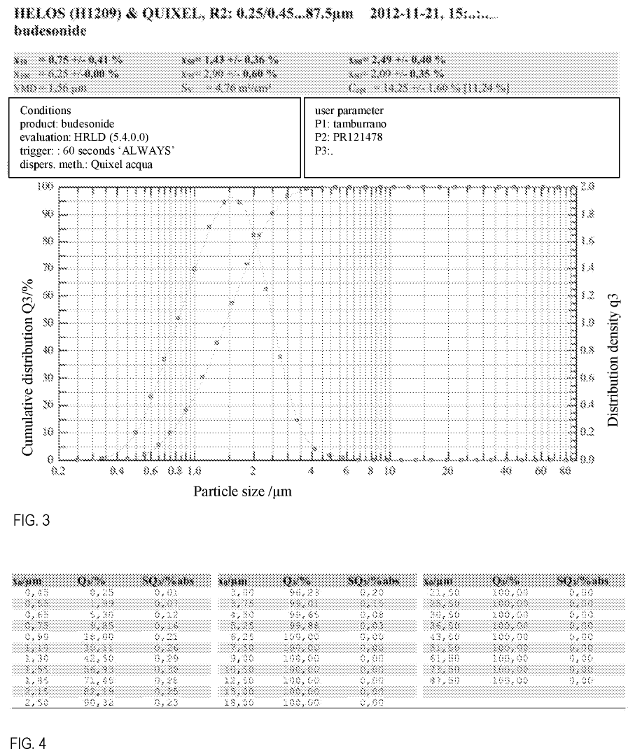

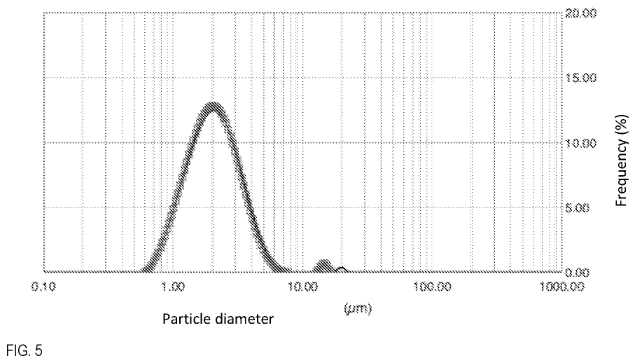

FIGS. 3 and 4 respectively show a diagram of the Cumulative Distribution Q3% (HELOS Particle Size Analysis) with respect to the particle size in μm, and a table corresponding to said diagram of the Cumulative Distribution with standard deviation, according to a possible embodiment of the budesonide that can be used in this invention. FIG. 4 shows that x0/μm=2,50 has a Q3% equal to 90,32 and that x0/μm=3.75 has a Q3% equal to 99,01. In this range of there is half the total weight, almost as in the remaining 100%;

FIG. 5 discloses a diagram of the average size distribution of a budesonide in the form of micronized powder, obtained by means of a laser diffractometer, according to another embodiment of the present invention.

The present invention relates to budesonide, or a salt thereof, or mixtures thereof for ophthalmic use in a method for the curative treatment of an eye disorder or ailment or disease in a subject in need, preferably wherein said method comprises an administration of said budesonide to said subject through the ocular topical route.

The disorder or said ailment or said disease is an inflammation of the ocular adnexa or an inflammation of the eyeball that can be of various origins: microbiological (virus and pathogens), mechanical or particle, or allergic.

Inflammation of the ocular adnexa is preferably selected from the group comprising or, alternatively, consisting of conjunctivitis, blepharitis, blepharoconjunctivitis, chalazion, dacryocystitis.

Inflammation of the eyeball is preferably selected from the group comprising or, alternatively, consisting of keratitis, keratoconjunctivitis, scleritis or episcleritis, uveitis.

More preferably, said disorder or said ailment or said disease is an eye inflammation selected from the group comprising, or alternatively, consisting of allergic conjunctivitis, allergic keratitis, keratitis, dry eye syndrome, allergy eye redness, and swelling from ocular phlogosis.

However, in the context of the present invention we consider that said budesonide, or salt thereof, or mixtures thereof, is used and administered in the form of an aqueous suspension composition given that said budesonide is in the form of a powder—in short, a powder in micronized form. Said budesonide in the form of a powder contains a micronized powder with an average size distribution of less than or equal to 20 μm (preferably between 0.05 μm and 20 μm) which, in turn, contains a fraction of an ultrafine micronized powder having an average size distribution less than or equal to 10 μm (preferably comprised from 0.1 μm to 10 μm).

Said budesonide is obtained and supplied by the manufacturer already in this form according to an average size distribution shown in the diagram of FIG. 3, and in tabular form in FIG. 4, which show the cumulative distribution Q3% with respect to the particle size in μm.

Preferably, said budesonide has an average size distribution shown in the diagram of FIG. 5, and in tabular form in Table A.1 below (in which the numerical values are expressed in μm).

TABLE A.1

| Dx(10) | Dx(50) | Dx(90) |
|---|---|---|
| 1.10 | 2.02 | 3.68 |

In Table A.1, the parameters Dx(10), Dx(50) and Dx(90) respectively indicate that 10%, 50% and 90% by weight of the particles analysed have a dimensional distribution below the numerical values (expressed in μm) indicated in such Table.

Said powder in the micronized form of budesonide preferably comprises—on average—substantially spherical-shaped powder particles.

In this description, the expression “micronized” means that substantially the entire particle population of said powder comprises particles having an average size distribution by number equal to or less than 20 μm, preferably comprised from 0.05 μm to 20 μm, more preferably comprised from 0.1 μm to 10 μm, even more preferably comprised from 0.2 μm to 5 μm, even more preferably comprised from 0.25 μm to 3.75 μm, even more preferably comprised from 0.40 μm to 3.0 μm.

In this description, “substantially the entire particle population” means that a total percentage by volume equal to or greater than 90%, i.e. comprised from 94% to 100%, preferably from 94% to 98%, of said powder particles has a distribution equal to or less than 20 μm, preferably comprised from 0.05 μm to 20 μm, more preferably comprised from 0.1 μm to 10 μm, even more preferably comprised from 0.2 μm to 5 μm, even further more preferably comprised from 0.25 μm to 3.75 μm, most preferably comprised from 0.40 μm to 3.0 μm.

By way of example, such average size distribution is determined according to the calculation of the mean geometric diameter for number determined with the aid of modern electronic equipment such as light scattering or laser. The same results can be obtained through visual observation and field count under the microscope.

For example, the average size distribution could be determined by means of a Spraytec laser diffractometer (Malvern Panalytics, UK), preferably using the instrumental operating parameters shown in Table A.2 below, where the parameters not specified in such table were maintained as per default specifications of the laser diffractometer.

TABLE A.2

| Parameter | Value | |
|---|---|---|
| Optical properties of | Cyclohexane | |
| the dispersing agent | (refraction index 1.43) | |
| Focal Lens | 300 | mm* |

| | |
|---|---|
| Detector Range | 10-last |
| Type of measurement | Time-based |
| Time of measurement | 1 minute, sampling each second |

*The lens allows to measure particles in the range comprised from 0.1 μm-900 μm (Dv₅₀ 0.5 μm-600 μm).

In the presence of asymmetric powder particles (with non-spherical symmetry), the so-called “equivalent spherical diameter”, which compares the particle size with the diameter of a sphere having the same volume, the same area, the same projection area and the same volume/surface ratio, is preferably used. The equivalent spherical diameter corrects or recalculates the size of a non-spherical particle according to the diameter of a sphere having the same volume.

Said average size distribution less than or equal to 20 μm (preferably comprised from 0.05 μm to 20 μm) does not show contact irritant effects, and it is sufficiently fine to avoid the perception of powder particles by the eye as a solid.

Said powder in micronized form of budesonide is preferably suspended in a liquid carrier, preferably an aqueous carrier, more preferably water, even more preferably purified water or water for injections.

Furthermore, forming an object of the present invention is a composition comprising a mixture comprising or, alternatively, consisting of:

(i) a budesonide, or a salt thereof, or mixtures thereof; and
(ii) at least one suspending/thickening agent, and optionally (iii) one or more physiologically and/or pharmacologically acceptable excipients;
for use in a method for the curative treatment of an inflammation of the ocular adnexa or the eyeball in a subject in need, wherein said method comprises an administration of said composition to said subject.

Furthermore, forming an object of the present invention is a composition comprising a mixture comprising or, alternatively, consisting of:

(i) a budesonide, or a salt thereof, or mixtures thereof, in form of micronized powder, wherein said micronized powder comprises powder particles having an average size distribution comprised from 0.05 μm to 20 μm;
(ii) at least one suspending/thickening agent selected from the group comprising, or alternatively, consisting of: cellulose, carboxymethylcellulose (CMC), sodium carboxymethylcellulose (Na-CMC), refined carboxymethylcelluloses, or mixtures thereof, wherein said composition comprises only the water-soluble fractions of CMC, Na-CMC and/or refined carboxymethylcelluloses, and optionally
(a.2) or more physiologically and/or pharmacologically acceptable excipient,

wherein said composition is for use in a method for the curative treatment of an inflammation of the ocular adnexa or an inflammation of the eyeball in a subject in need.

The average particle size distribution of the powder particles comprised from 0.05 μm to 20 μm, preferably comprised from 0.1 μm to 10 μm, more preferably comprised from 0.2 μm to 5 μm, even more preferably comprised from 0.25 μm to 3.75 μm, more preferably comprised from 0.40 μm to 3.0 μm, relates to the only material in suspension detectable within said composition, which is preferably only budesonide. As a matter of fact, said composition is preferably devoid of opaque ingredients.

In the present description, the term “opaque ingredients” is used to indicate additional ingredients with respect to budesonide, or salt thereof, or mixtures thereof which, suspended in said composition, could render said composition less transparent to visible light with respect to a composition devoid of said ingredients. The opaque ingredients are thus ingredients suspended and undissolved in said composition, and additionally with respect to budesonide, or salt thereof, or mixtures thereof.

In other words, said composition comprises, additionally to (i), (ii) and one or more optional (iii) physiologically and/or pharmacologically acceptable excipients dissolved in solution which, since solubilised, do not have an opacifying effect on the composition and they do not generate contact irritant effects.

In the context of the present invention, the term “composition/s” is used to indicate a pharmaceutical composition, or a medical device composition.

In an embodiment, said composition is a liquid composition, preferably in the form of a suspension in water, more preferably purified water or water for injections. More preferably, said suspension has a pH value comprised from 6±0,1 to 8±0,1, preferably comprised from 6,5±0,1 to 7,5±0,1, even more preferably from 6.7±0,1 to 7,3±0,1 (at 20° C. and 1 Atm).

Said method comprises an administration of said composition through the ocular topical route, preferably on the ocular adnexa or on the eyeball, more preferably on the conjunctiva and cornea, even more preferably on the cornea (with eye open).

An amount of said (i) budesonide, or salt thereof, or mixtures thereof is comprised from 0,005% (w/v) to 3% (w/v) by weight, with respect to a total volume of said liquid composition; preferably comprised from 0,01% (w/v) to 2% (w/v) by weight; even more preferably comprised from 0.05% (w/v) to 1,5% (w/v) by weight; for example 0.1% (w/v), 0,2% (w/v), 0,3% (w/v), 0,4% (w/v), 0.5% (w/v), 0,6% (w/v), 0,7% (w/v), 0,8% (w/v), 0,9% (w/v) or 1% (w/v). Even more preferably, the amount of said (i) budesonide, or salt thereof, or mixtures thereof in said liquid composition is comprised from 0.05% (w/v) to 0,2% (w/v), preferably comprised from 0,07% (w/v) to 0,15% (w/v), even more preferably comprised from 0,08% (w/v) to 0,12% (w/v).

By way of example, an amount from 0.05 g to 1,5 g, for example 0,1 g, 0,2 g, 0,3 g, 0,4 g, 0,5 g, 0,6 g, 0,7 g, 0,8 g, 0,9 g or 1,0 g of said budesonide (i), or salt thereof, or mixtures thereof could be suspended in 100 ml of the liquid of said liquid composition.

The use of a low dose/concentration of budesonide powder in micronized form in the composition subject of the present invention (containing or consisting of a micronized powder with an average size distribution less than or equal to 20 μm (preferably comprised from 0.05 μm to 20 μm) and/or an ultrafine micronized powder with an average size distribution less than or equal to 10 μm (preferably comprised between 0.1 μm and 10 μm)) comprised from 0,01% (w/v) to 0,3% (w/v) by weight, preferably comprised from 0.05% (w/v) to 0,2% (w/v) by weight, even more preferably comprised from 0,08% (w/v) to 0,12% (w/v), for example 0.1% (w/v) by weight, allows to have a smaller micro-particle fraction in suspension and, thus, budesonide can be suspended more effectively by the suspending/thickening agent. Thus, a smaller amount of suspending/thickening agent necessary to suspend a low dose of budesonide powder in micronized form, comprised from those mentioned above, makes a drop of composition of the present invention in the form of a suspension being applied less viscous (preferably a viscosity comprised from 10 CPS to 25 CPS measured at 20° C. and 1 Atmosphere of ambient pressure, more preferably comprised from 15 CPS to 25 CPS, for example 11 CPS, 13 CPS, 15 CPS, 17 CPS, or 19 CPS) and more spreadable.

In the composition of the present invention, said (ii) at least one suspending/thickening agent used in combination with the (i) budesonide, or a salt thereof, or mixtures thereof, is selected from the group comprising or, alternatively, consisting of: cellulose, carboxymethylcellulose (CMC), sodium carboxymethylcellulose (Na-CMC), refined carboxymethylcelluloses, or mixtures thereof, preferably in cross-linked form/s.

Said suspending/thickening agent is present in said mixture at an amount by weight comprised from 0,005% (w/v) to 3% (w/v) by weight, with respect to a total volume of said composition; preferably at an amount by weight comprised from 0,01% (w/v) to 2% (w/v) by weight; more preferably comprised from 0,05% (w/v) to 1,5% (w/v) by weight; even more preferably comprised from 0.1% (w/v) to 1% (w/v); for example 0,12% (w/v), 0,14% (w/v), 0,16% (w/v), 0,18% (w/v), 0,20% (w/v), 0,25% (w/v), 0,30% (w/v), 0,4% (w/v), 0.5% (w/v), 0,6% (w/v), 0.7% (w/v), 0,8% (w/v), 0,9% (w/v), or 1% (w/v).

The (ii) suspending/thickening agent, used in combination with the component (i) of the composition of the present invention, is selected from cellulose polymers having only the water-soluble fractions of carboxymethylcellulose (CMC), sodium carboxymethylcellulose (Na-CMC) and/or refined carboxymethylcelluloses. These cellulose polymers are transparent (i.e., non-opaque) when dissolved in the liquid carrier, for example water, since they do not contain solid residues and they can be fully gelled in water without suspended particles with an average size distribution of component (i) greater than 20 μm being irritating to the eye.

The cellulose polymers having only the water-soluble fractions of carboxymethylcellulose (CMC), sodium carboxymethylcellulose (Na-CMC) and/or refined carboxymethylcelluloses have a minimum carboxymethylcellulose content comprised from 97% to 100% by weight, preferably from 98% to 99,9% by weight, in water at 25° C. and 1 atmosphere pressure. Said water-soluble fractions of CMC, Na-CMC and/or refined carboxymethylcelluloses preferably have an amount of sodium comprised from 7,0% to 9,5% by weight, more preferably comprised from 7,5% to 9,3% by weight, even more preferably comprised from 8,2% to 9% by weight. Said water-soluble fractions of carboxymethylcellulose (CMC), sodium carboxymethylcellulose (Na-CMC) and/or refined carboxymethylcelluloses carboxymethylcellulose have a bulk density comprised from 0.45 g/cm³ to 1.05 g/cm³, preferably comprised from 0.55 g/cm³ to 1.00 g/cm³ (measured according to known techniques and equipment).

Said at least one agent (ii) suspending/thickening agent, used in combination with the component (i) in the liquid composition of the present invention, is—as mentioned—selected from cellulose polymers having only the water-soluble fractions of carboxymethylcellulose (CMC), sodium carboxymethylcellulose (Na-CMC) and/or refined carboxymethylcellulose. These cellulose polymers have the advantage of controlling and delaying the delivery of (i) budesonide, or salt thereof, or mixtures thereof at the interface with cellular tissues, since only the water-soluble fractions of refined CMC, Na-CMC and/or refined carboxymethylcelluloses act as a water-soluble matrix for budesonide.

Such delivery delay is particularly marked when budesonide is in form of micronized powder comprising powder particles having an average size distribution comprised from 0.05 μm to 20 μm, preferably comprised from 0.1 μm to 10 μm, more preferably comprised from 0.2 μm to 5 μm, even more preferably comprised from 0.25 μm to 3.75 μm, more preferably comprised from 0.40 μm to 3.0 μm.

Cellulose polymers having only the water-soluble fractions of carboxymethylcellulose (CMC), sodium carboxymethylcellulose (Na-CMC) and/or refined carboxymethylcelluloses exclude products containing or, alternatively, consisting of microcrystalline cellulose and sodium carboxymethylcellulose at variable ratios and having non-water-soluble fractions. Such products are known under the trade names Avicel® RC581, Avicel® RC591 and Avicel® CL611. By way of example, the safety data sheet of the product Avicel® CL611 indicates, with respect to solubility in water, that such product may be dispersed (but not dissolved) in water.

Refined carboxymethylcelluloses (for example that manufactured by Blanose™) is a purified salt of sodium carboxymethylcellulose (Na-CMC) with a minimum carboxymethylcellulose content (CMC) content comprised from 95% to 99,9%, preferably comprised from 96% to 98%. Refined Na-CMC is a water-soluble anionic polymer which acts as suspending/thickening agent in the composition of the present invention. Refined Na-CMC may have two degrees of substitution (DS) and some degrees of viscosity. The degree of substitution (DS) is defined as the average number of hydroxyl groups substituted with anhydroglucose units (AGU) in said refined CMC.

Typical substitution levels and viscosity ranges are indicated below. Generally, higher levels of substitution enhance compatibility with other components of the composition/system. More precisely, higher substitution levels enhance the water solubility of the refined Na-CMC.

TABLE 1
Degree of substitution. Method: MA 304.1506°

| Type of DS | Limits of substitution | Sodium content |
|---|---|---|
| 7 (^{a}) | From 0.65 to 0.90 | 7.0-8.9 |
| | From 0.80 to 0.95 | 8.2-9.3 |

(^{a}) some degrees have a narrower substitution range

Therefore, the refined CMC in said composition preferably has a substitution limit comprised from 0,65 to 0,90 or from 0,80 to 0,95, more preferably comprised from 0,80 to 0,95.

TABLE 2
Typical viscosity ranges. Method MA 304.1001A

| | | | | Brookfield LVF | |
|---|---|---|---|---|---|
| DS7 | DS9 | Concen- | Range 25° C. | settings | |
| Degrees | Degrees | trations | mPa-s | Ago n. | tpm |
| 7H9 | | 1 | 4.000-9.000 | 4 | 30 |
| 7H4 | 9H4 | 1 | 2.500-4.500 | 4 | 30 |
| 7H | | 1 | 1..500-2.500 | 3 | 30 |
| 7M65 | | 2 | 3.000-6.500 | 4 | 30 |
| 7M31 | | 2 | 1.500-3.100 | 3 | 30 |
| 7M | | 2 | 300-600 | 2 | 30 |
| 7M2 | 9M2 | 2 | 100-200 | 2 | 60 |
| 7M1 | | 2 | 50-100 | 1 | 60 |
| 7L | | 2 | 25-50 | 1 | 60 |
| 7L2 | | 6 | 120-280 | 2 | 60 |
| 7L1 | | 6 | 90-130 | 2 | 60 |
| 7EL | | 6 | 35-60 | 1 | 60 |
| 7UL | | 6 | 10-25 | 1 | 60 |

This table indicates the available viscosity levels.

Other typical properties of refined CMC (Table 3):

TABLE 3

| Properties | Limits | | Method |
|---|---|---|---|
| Purity, 100 − | 98 | min. | MA 304.1501A/1502A |
| NaCI + Na glycolate), % | | | |
| Bulk density, g/cm³ | 0.55-1.00 | | MA 304.1607A |
| Humidity at packaging, % | 8 | max. | MA 304.1600A |

| | | |
|---|---|---|
| pH of the solutions, | | MA 304.1004A |
| All types L, at 2% | 5.0-8.5 | |
| All types M, at 1% | 6.5-8.5 | |
| All types M, at 1% | 6.5-8.5 | |

Size gradings of refined CMC. Method 304.1602A (Table 4):

TABLE 4

| Desig- | Descrip- | | | Opening of the sieve | |
|---|---|---|---|---|---|
| nation | tion | Limitation | | mm | ASTM n. |
| X | Thin | 0.5% max. | MAINTAINED | 0.250 | 60 |
| | | 80% mon. | PAST | 0.075 | 200 |
| — | Normal | 1% max. | MAINTAINED | 0.600 | 30 |
| | | 10% max. | MAINTAINED | 0.425 | 40 |
| C | Granulate | 0% | MAINTAINED | 1.70 | 12 |
| | | 50% max. | MAINTAINED | 0.85 | 20 |
| | | 50% max. | PAST | 0.425 | 40 |
| | | 15% max. | PAST | 0.18 | 80 |
| C1 | Granulate | 0% | MAINTAINED | 1.70 | 12 |
| | | 50% max. | MAINTAINED | 0.85 | 20 |
| | | 50% max. | PAST | 0.18 | 80 |
| C2 | Granulate | 0% | MAINTAINED | 1.70 | 12 |
| | | 10% max. | PAST | 0.18 | 80 |

The Na-CMC used in said composition preferably has CAS No. 9004-32-4 and it is devoid of microcrystalline cellulose (CAS No. 9004-34-6).

Besides (i) and (ii), the composition of the present invention further comprises or consists of optional (iii) physiologically and/or pharmacologically acceptable excipients which may preferably contribute to formulating the composition in the form of an aqueous suspension for topical use, for example as eye drops.

The physiologically and/or pharmacologically acceptable excipients are selected from the group comprising or, alternatively, consisting of: a preservative, preferably potassium sorbate and/or benzalkonium chloride, a surfactant, preferably polyoxyethylene (20) sorbitan monooleate (Polysorbate 80), a chelating agent, preferably ethylenediaminetetraacetic acid (EDTA), or a salt thereof, or mixtures thereof, an isotonic agent, preferably mannitol or sodium chloride, a carrier, preferably an aqueous carrier, more preferably water, or mixtures thereof.

In an embodiment, the composition of the present invention may comprise:

(i) a budesonide, or a salt thereof, or mixtures thereof;
(ii) at least one suspending/thickening agent selected from cellulose, carboxymethylcellulose (CMC), sodium carboxymethylcellulose (Na-CMC), refined carboxymethylcellulose, or mixtures thereof;
(iii.a) potassium sorbate (preservative);
(iii.b) polyoxymethylene (20) sorbitan monooleate (Polysorbate 80) (surfactant);
(iii.c) ethylenediaminetetraacetic acid (EDTA), or a salt thereof, or mixtures thereof (sequestrant);
(iii.d) mannitol or sodium chloride (tonicity agent);
(iii.e) benzalkonium chloride (preservative);
(iii.f) water, preferably purified water or water for injections (liquid carrier).

In an embodiment, the composition of the present invention may comprise the following amounts expressed as weight with respect to total volume of said composition (w/v):

(i) budesonide, or a salt thereof, or mixtures thereof at an amount comprised from 0,01% (w/v) to 0,3% (w/v) by weight, preferably comprised from 0,07% (w/v) to 0,2% (w/v) by weight, even more preferably comprised from 0,08% (w/v) to 0,15% (w/v), for example 0.1% (w/v) by weight;
(ii) cellulose, carboxymethylcellulose (CMC), sodium carboxymethylcellulose (Na-CMC), refined carboxymethylcellulose, or mixtures thereof, at an amount comprised from 0.05% (w/v) to 1,5% (w/v) by weight; preferably comprised from 0.1% (w/v) to 1% (w/v); for example 0,12% (w/v), 0,14% (w/v), 0,16% (w/v), 0,18% (w/v), 0,20% (w/v), 0,25% (w/v), 0,30% (w/v), 0,4% (w/v), 0.5% (w/v), 0,6% (w/v), 0,7% (w/v), 0,8% (w/v), 0,9% (w/v), or 1% (w/v);
(iii.a) potassium sorbate at an amount comprised from 0,005% (w/v) to 3% (w/v), preferably comprised from 0,01% (w/v) to 2% (w/v), more preferably comprised from 0.05% (w/v) to 1,5% (w/v), for example 0,100% (w/v);
(iii.b) polyoxymethylene (20) sorbitan monooleate (Polysorbate 80) at an amount comprised from 0.001% (w/v) to 2% (w/v), preferably comprised from 0,005% (w/v) to 1% (w/v), even more preferably comprised from 0,01% (w/v) to 1,5% (w/v), for example 0,030% (w/v);
(iii.c) ethylenediaminetetraacetic acid (EDTA), or a salt thereof, or mixtures thereof at an amount comprised from 0,005% (w/v) to 3% (w/v), preferably comprised from 0,01% (w/v) a 2% (w/v), more preferably comprised from 0.05% (w/v) to 1,5% (w/v), for example 0,100% (w/v);
(iii.d) mannitol or sodium chloride, preferably mannitol, at an amount comprised from 0,01% (w/v) to 10% (w/v), preferably comprised from 0.1% (w/v) to 7% (w/v), more preferably comprised from 0.5% (w/v) to 5% (w/v), even more preferably comprised from 0,8% (w/v) to 3% (w/v), for example 1,800% (w/v);
(iii.e) benzalkonium chloride at an amount comprised from 0,005% (w/v) to 3% (w/v), preferably comprised from 0,01% (w/v) to 2% (w/v), more preferably comprised from 0.05% (w/v) to 1,5% (w/v), for example 0,100% (w/v);
(iii. f) water, preferably purified water or water for injections, up to a volume of 10 ml of said composition.

Furthermore, forming an object of the present invention is a method for the curative treatment of an inflammation of the ocular adnexa or inflammation of the eyeball in a subject in need, wherein said treatment method comprises administering said composition to said subject.

In an embodiment, the administration of from 1 drop to 4 drops, from 1 to 3 times a day, of a composition from 0.1% to 0,5%, preferably 0,2% (0,2 g/100 ml).

### EXAMPLES

### Example 1: Preparation of Compositions According to the Present Invention

Two different liquid compositions subject of the present invention were prepared, each with a different dose of budesonide: 1) a first dose at 0,10% w/v (N641T-A/L), and 2) a second high dose at 0,20% w/v (N641T-A/M)(N64IT-A/H; the high dose did not appear in the current FIG. 2, nor in the PGE2 diagram. For homogeneity we decided to omit the high dose data).

Such two liquid compositions have the qualitative and quantitative composition (expressed in % w/v) reported in Table 5A below.

TABLE 5A

| | | Composition | |
|---|---|---|---|
| | | N64IT- | N64IT- |
| Component | Function | A/L (%) | A/M (%) |
| Micronized | Active | 0.100 | 0.200 |
| budesonide | | | |
| Polysorbate 80 | Surfactant | 0.030 | 0.030 |
| Disodium-EDTA | Chelating agent | 0.010 | 0.010 |
| Sodium-CMC | Suspending/thickening | 0.160 | 0.160 |
| | agent | | |
| Mannitol | Isotonic agent | 1.800 | 1.800 |
| Benzalkonium | Preservative | 0.010 | 0.010 |
| chloride | | | |

| | | |
|---|---|---|
| Purified water | Liquid solvent/carrier | q.s. up to10 ml |

Therefore, the concentration of budesonide is the only variable component in said two compositions.

The storage conditions and the period of stability before/after opening of containers containing the compositions are respectively:

before opening: room temperature at 20° C.; 6 months;
after opening: 4° C.; 8 weeks.

Table 5B below reports pH, viscosity (measured at 20° C.) and osmolality values of the placebo composition discussed hereinafter in Table 5B, and of the compositions N641T-A/L and N641T-A/M of Table 5A above.

TABLE 5B

| Composition | pH | Viscosity (cPs) | Osmolality (mOsm) |
|---|---|---|---|
| N64IT-V (placebo) | 6.6 | 15 | 270 |
| N64IT-A/L (low dose) | 6.7 | 15 | 271 |
| N64IT-A/M (medium dose) | 6.6 | 14 | 275 |
| N64IT-A/H (high dose) | 6.6 | 18 | 277 |

### Example 2: Tests on the Efficacy of the Compositions Prepared in Example 1 on the Animal

The first (N641T-A/L) and the second (N641T-A/M) liquid composition of budesonide of Example 1 were used for an ophthalmic efficacy test on the animal (rabbit) according to the following trial protocol. The protocol chosen to induce eye inflammation in the animal and to evaluate the efficacy of the substances administered as anti-inflammatory provides for an initial administration 6 hours before the inflammatory event (tissue injury, at time t=0), and observations temporally after 30 minutes, 1 hour and 2 hours of the evolution of the inflammation symptoms, according to the scheme of Table 6 below.

TABLE 6
Experimental scheme: 6 animals per time point

| | −6 | | +30 | +1 | +2 |
|---|---|---|---|---|---|
| Time | hours | t = 0 | min | hour | hours |
| Induction: collection of | | X | | | |
| 200 μl of aqueous humour | | | | | |
| Maxidex ® (0.1% Dexamethasone) | X | | | | |
| Lotemax ® (0.5% Loteprednol) | X | | | | |
| N64IT-V (placebo) | X | | | | |
| N64IT-A/L (first dose) | X | | | | |
| N64IT-A/M (second dose) | X | | | | |
| Slit lamp eye examination | | | X | X | X |
| Protein concentration assay | | | X | X | X |
| in aqueous humour | | | | | |
| PGE2 concentration assay | | | X | X | X |
| in aqueous humour | | | | | |

The efficacy trial protocol provided for: pre-treatment with the two budesonide compositions at different doses, pre-treatments in other groups of subjects with a placebo (N641T-V; liquid carrier) and pre-treatment with two market reference products based on different corticosteroids (Maxidex® containing 0,1% dexamethasone; Lotemax® containing 0.5% loteprednol), for eye use.

In the efficacy evaluation (30 minutes, 1 hour and 2 hours after the inflammatory event), three different types of observations were conducted on the 6 animals per treatment group, aimed at establishing decrease in the inflammatory condition induced by a standard paracentesis (incision) of the corneal tissue. The first observations relate to possible material suspended in the aqueous humour deriving from the inflammatory process determined by means of a slit lamp, used in the ophthalmic field to evaluate the anatomical and functional condition of the eye. According to this observation, the lower the presence of suspended material (corpuscles) in aqueous humour, the lower the detected condition of inflammation. The results of the first observations are reported in graphical form in the attached FIG. 1, after 2 hours. Only this data is reported for solely for explanatory purposes.

In these figures, the column diagrams relating to each pre-treatment were reported, where such diagrams report—on the abscissa—a mean inflammation index (parameter of integer value comprised from 0 to 4) relative to the only eye of the animal in which the inflammation was induced.

From FIG. 1 it can be inferred that, surprisingly, the first dose of the budesonide liquid composition (0,10% w/v; N641T-A/L composition) is the most effective by virtue of a smaller amount of suspended corpuscles (flares). This greater efficacy is manifested both in comparison with all the other substances (placebo, carrier or cortisone agents other than budesonide) with which the pre-treatment was carried out, and in comparison with the second dose (0,20% w/v; N641T-A/M) of the composition comprising budesonide.

The latter consideration is particularly unexpected, since a low dose could be expected to reveal an equivalent but not greater efficacy with respect to a higher dose.

The data of a second and a third test typical of the inflammatory phenomena on amounts of proteins and prostaglandins PGE2 in aqueous humour, in which these amounts are both direct indices of the inflammatory conditions in a tissue, were therefore evaluated: the higher the concentration of proteins and of PGE2, the more inflammatory conditions are present in said tissue.

The results of the second observations relating to protein concentration are reported in graphical form in the attached FIG. 2 after 2 hours. Since the PGE2 concentration values are in line with the protein values, a separate diagram relating to PGE2 at 2 hours is not reported in the attached figures. In these figures, the column diagrams relating to each pre-treatment were reported, where such diagrams report—on the ordinate—a protein concentration (expressed in ng/ml, nanograms of proteins per millilitre of aqueous humour) relative to the only eye of the animal in which the inflammation was induced. A comparison between the two doses of the compositions subject of the present invention shows a substantial equivalence of effectiveness.

A comparison between the compositions subject of the present invention and the other compositions tested once again shows a better and significant effectiveness of the compositions subject of the invention.

This better activity of the compositions subject of the present invention is particularly marked, especially after 2 hours of tissue injury.

Embodiments (En) of the present invention are illustrated below:

E1. A budesonide, or a salt thereof, or mixtures thereof for ophthalmic use in a method for the curative treatment of an eye disorder or ailment or disease in a subject in need.

E2. Budesonide, or a salt thereof, or mixtures thereof for use according to E1, wherein said budesonide is administered to said subject through the ocular topical route.

E3. Budesonide, or a salt thereof, or mixtures thereof according to E1 or E2, wherein said budesonide is in the form of micronized powder, wherein said micronized powder comprises powder particles having an average size distribution comprised from 0.05 μm to 20 μm, preferably comprised from 0.1 μm to 10 μm, or comprised from 0.2 μm to 5 μm, more preferably comprised from 0.05 μm to 5 μm, even more preferably comprised from 0.25 μm to 1 μm.

E4. Budesonide, or a salt thereof, or mixtures thereof for use according to any one from E1-E3, wherein said disorder, or said ailment, or said disease is an inflammation of the ocular adnexa or an inflammation of the eyeball.

E5. Budesonide, or a salt thereof, or mixtures thereof for use according to E4, wherein said inflammation of the ocular adnexa is selected from the group comprising or, alternatively, consisting of conjunctivitis, blepharitis, blepharoconjunctivitis, chalazion, dacryocystitis.

E6. Budesonide, or a salt thereof, or mixtures thereof for use according to E4-E5, wherein said inflammation of the eyeball is selected from the group comprising or, alternatively, consisting of keratitis, keratoconjunctivitis, scleritis or episcleritis, uveitis.

E7. Budesonide, or a salt thereof, or mixtures thereof for use according to any one from E4-E6, wherein said inflammation is of allergic origin.

E8. Budesonide, or a salt thereof, or mixtures thereof for use according to any one from E1-E7, when dependent on E3, wherein said micronized powder is suspended in a liquid carrier.

E9. Budesonide, or a salt thereof, or mixtures thereof for use according to E8, wherein said liquid carrier is an aqueous carrier, preferably water, even more preferably purified water or water for injections.

Embodiments (Fn) of the present invention are illustrated below:

F1. A composition comprising a mixture comprising, or alternatively, consisting of:

(i) a budesonide, or a salt thereof, or mixtures thereof;
(ii) at least one suspending/thickening agent, and optionally (iii) one or more physiologically and/or pharmacologically acceptable excipients, wherein said composition is for use in a method for the curative treatment of an inflammation of the ocular adnexa or an inflammation of the eyeball in a subject in need.

F2. Composition for use according to F1, wherein an amount of said (i) budesonide, or salt thereof, or mixtures thereof is comprised from 0,005% (w/v) to 3% (w/v) by weight with respect to a total volume of said composition, preferably comprised from 0,01% (w/v) to 2% (w/v), even more preferably comprised from 0.05% (w/v) to 1,5% (w/v), for example 0.1% (w/v), 0,2% (w/v) or 0.5% (w/v).

F3. Composition for use according to any one from F1-F2, wherein said (ii) suspending/thickening agent is selected from the group comprising or, alternatively, consisting of: cellulose, carboxymethylcellulose (CMC), sodium carboxymethylcellulose (Na-CMC), refined carboxymethylcelluloses, or mixtures thereof or mixtures thereof, preferably crosslinked.

F4. Composition for use according to any one from F1-F3 wherein said one or more physiologically and/or pharmacologically acceptable excipients is selected from the group comprising or, alternatively, consisting of: a preservative, a surfactant, a chelating agent, an isotonic agent, a carrier, or mixtures thereof.

F5. Composition for use according to any one from F1-F4, comprising:

(i) a budesonide, or a salt thereof, or mixtures thereof;
(ii) cellulose, carboxymethylcellulose (CMC), sodium carboxymethylcellulose (Na-CMC), refined carboxymethylcelluloses, preferably in cross-linked form/s, or mixtures thereof;
(iii.a) potassium sorbate;
(iii.b) polyoxyethylene (20) sorbitan monooleate (Polysorbate 80);
(iii.c) ethylenediaminetetraacetic acid (EDTA), or a salt thereof, or mixtures thereof;
(iii.d) mannitol;
(iii.e) benzalkonium chloride;
(iii.f) water, preferably purified water or water for injections;

preferably said composition being free of opaque ingredients.

F6. Composition for use according to F5, comprising (amounts expressed as weight with respect to the total volume of said composition; w/v):

(i) a budesonide, or a salt thereof, or mixtures thereof comprised from 0,005% (w/v) to 3% (w/v), preferably comprised from 0,01% (w/v) to 2% (w/v), even more preferably comprised from 0.05% (w/v) to 1,5% (w/v), for example 0.1% (w/v), 0,2% (w/v) or 0.5% (w/v);
(ii) cellulose, carboxymethylcellulose (CMC), sodium carboxymethylcellulose (Na-CMC), refined carboxymethylcelluloses preferably in cross-linked form/s, or mixtures thereof comprised from 0,005% (w/v) to 3% (w/v), preferably comprised from 0,01% (w/v) to 2% (w/v), more preferably comprised from 0,01% (w/v) to 2% (w/v) even more preferably comprised from 0.05% (w/v) to 1,5% (w/v), for example 0,160% (w/v);
(iii.a) potassium sorbate comprised from 0,005% (w/v) to 3% (w/v), preferably comprised from 0,01% (w/v) to 2% (w/v), more preferably comprised from 0.05% (w/v) to 1,5% (w/v), for example 0,100% (w/v);
(iii.b) polyoxyethylene (20) sorbitan monooleate (Polysorbate 80) comprised from 0.001% (w/v) to 2% (w/v), preferably comprised from 0,005% (w/v) to 1% (w/v), even more preferably comprised from 0,01% (w/v) to 1,5% (w/v), for example 0,030% (w/v);
(iii.c) ethylenediaminetetraacetic acid (EDTA), or a salt thereof, or mixtures thereof comprised from 0,005% (w/v) to 3% (w/v), preferably comprised from 0,01% (w/v) to 2% (w/v), more preferably comprised from 0.05% (w/v) to 1,5% (w/v), for example 0,100% (w/v);
(iii.d) mannitol comprised from 0,01% (w/v) to 10% (w/v), preferably comprised from 0.1% (w/v) to 7% (w/v), more preferably comprised from 0.5% (w/v) to 5% (w/v), even more preferably comprised from 0,8% (w/v) to 3% (w/v), for example 1,800% (w/v);
(iii.e) benzalkonium chloride comprised from 0,005% (w/v) to 3% (w/v), preferably comprised from 0,01% (w/v) to 2% (w/v), more preferably comprised from 0.05% (w/v) to 1,5% (w/v), for example 0,100% (w/v);
(iii. f) water, preferably purified water or water for injections, up to a volume of 10 ml of said composition.

F7. Composition for use according to any one from F4-F6, comprising only the water-soluble fractions of CMC, Na-CMC and/or refined carboxymethylcelluloses.

F8. Composition for use according to any one from F1-F7, wherein said composition is a suspension in water, preferably wherein said suspension has a pH value (at 20° C. and 1 Atm) comprised from 6±0,1 to 8±0,1, preferably comprised from 6,5±0,1 to 7,5±0,1, even more preferably from 6.7±0,1 to 7,3±0,1.

F9. Composition for use according to any one from F1-F8, wherein said method comprises an administration of said composition through the ocular topical route, preferably on the ocular adnexa or on the eyeball, more preferably on the eyelid, conjunctiva, cornea and/or vitreous, even more preferably on the cornea (with the eye open) or on the eyelid (with the eye closed).

Embodiments (Gn) of the present invention are illustrated below:

G1. A composition comprising a mixture comprising, or alternatively, consisting of:

(i) a budesonide, or a salt thereof, or mixtures thereof, in form of micronized powder, wherein said micronized powder comprises powder particles having an average size distribution comprised from 0.05 μm to 20 μm;
(ii) at least one suspending/thickening agent selected from the group comprising, or alternatively, consisting of: cellulose, carboxymethylcellulose (CMC), sodium carboxymethylcellulose (Na-CMC), refined carboxymethylcelluloses, or mixtures thereof, wherein said composition comprises only the water-soluble fractions of CMC, Na-CMC and/or refined carboxymethylcelluloses, and optionally
(a.2) or more physiologically and/or pharmacologically acceptable excipient,

wherein said composition is for use in a method for the curative treatment of an inflammation of the ocular adnexa or an inflammation of the eyeball in a subject in need.

G2. The composition for use according to G1, wherein an amount of said (i) budesonide, or salt thereof, or mixtures thereof is comprised from 0,005% (w/v) to 3% (w/v) by weight with respect to a total volume of said composition, preferably comprised from 0,01% (w/v) to 2% (w/v), even more preferably comprised from 0.05% (w/v) to 1,5% (w/v), for example 0.1% (w/v), 0,2% (w/v) or 0.5% (w/v).

G3. The composition for use according to any of G1-G2, wherein said refined carboxymethylcelluloses have a degree of substitution (DS), defined as the average number of hydroxyl groups substituted with anhydroglucose units in said refined CMGs, comprised from 0,65 to 0,90 or comprised from 0,80 to 0,95, preferably comprised from 0,80 to 0,95.

G4. The composition for use according to any one of G1-G3, wherein said one or more physiologically and/or pharmacologically acceptable excipients is selected from the group comprising or, alternatively, consisting of: A preservative, preferably potassium sorbate and/or benzalkonium chloride, a surfactant, preferably polyoxyethylene (20) sorbitan monooleate, a chelating agent, preferably ethylenediaminetetraacetic acid (EDTA), or a salt thereof, or mixtures thereof, an isotonic agent, preferably mannitol or sodium chloride, a carrier, preferably an aqueous carrier, more preferably water, or mixtures thereof.

G5. The composition for use according to any one of G1-G4, comprising:

(i) a budesonide, or a salt thereof, or mixtures thereof;
(ii) cellulose, carboxymethylcellulose (CMC), sodium carboxymethylcellulose (Na-CMC), and/or refined carboxymethylcelluloses, preferably in cross-linked form/s, or mixtures thereof;
(iii.a) potassium sorbate;
(iii.b) polyoxyethylene (20) sorbitan monooleate (Polysorbate 80);
(iii.c) ethylenediaminetetraacetic acid (EDTA), or a salt thereof, or mixtures thereof;
(iii.d) mannitol or sodium chloride;
(iii.e) benzalkonium chloride;
(iii.f) water, preferably purified water or water for injections;

preferably, said composition being devoid of opaque ingredients, i.e., ingredients additional to said (i) budesonide, or salt thereof, or mixtures thereof suspended in said composition.

G6. The composition for use according to G5, comprising (amounts expressed as weight with respect to the total volume of said composition; w/v):

(i) a budesonide, or a salt thereof, or mixtures thereof comprised from 0,005% (w/v) to 3% (w/v), preferably comprised from 0,01% (w/v) to 2% (w/v), even more preferably comprised from 0.05% (w/v) to 1,5% (w/v), for example 0.1% (w/v), 0,2% (w/v) or 0.5% (w/v);
(ii) cellulose, carboxymethylcellulose (CMC), sodium carboxymethylcellulose (Na-CMC), refined carboxymethylcelluloses preferably in cross-linked form/s, or mixtures thereof comprised from 0,005% (w/v) to 3% (w/v), preferably comprised from 0,01% (w/v) to 2% (w/v), more preferably comprised from 0,01% (w/v) to 2% (w/v) even more preferably comprised from 0.05% (w/v) to 1,5% (w/v), for example 0,160% (w/v);
(iii.a) potassium sorbate comprised from 0,005% (w/v) to 3% (w/v), preferably comprised from 0,01% (w/v) to 2% (w/v), more preferably comprised from 0.05% (w/v) to 1,5% (w/v), for example 0,100% (w/v);
(iii.b) polyoxyethylene (20) sorbitan monooleate (Polysorbate 80) comprised from 0.001% (w/v) to 2% (w/v), preferably comprised from 0,005% (w/v) to 1% (w/v), even more preferably comprised from 0,01% (w/v) to 1,5% (w/v), for example 0,030% (w/v);
(iii.c) ethylenediaminetetraacetic acid (EDTA), or a salt thereof, or mixtures thereof comprised from 0,005% (w/v) to 3% (w/v), preferably comprised from 0,01% (w/v) to 2% (w/v), more preferably comprised from 0.05% (w/v) to 1,5% (w/v), for example 0,100% (w/v);
(iii.d) mannitol or sodium chloride comprised from 0,01% (w/v) to 10% (w/v), preferably comprised from 0,1% (w/v) to 7% (w/v), more preferably comprised from 0.5% (w/v) to 5% (w/v), even more preferably comprised from 0,8% (w/v) to 3% (w/v), for example 1,800% (w/v);
(iii.e) benzalkonium chloride comprised from 0,005% (w/v) to 3% (w/v), preferably comprised from 0,01% (w/v) to 2% (w/v), more preferably comprised from 0.05% (w/v) to 1,5% (w/v), for example 0,100% (w/v);
(iii. f) water, preferably purified water or water for injections, up to a volume of 10 ml of said composition.

G7. The composition for use according to any one of G1-G6, wherein said micronized powder comprises powder particles having an average size distribution comprised from 0.1 μm to 10 μm, preferably comprised from 0.2 μm to 5 μm, more preferably comprised from 0.25 μm to 3.75 μm.

G8. The composition for use according to any one of G1-G7, wherein said micronized powder comprises powder particles having an average size distribution comprised from 0.40 μm to 3.0 μm.

G9. The composition for use according to any one of G1-G8, wherein said composition is a suspension in water, preferably wherein said suspension has a pH value (at 20° C. and 1 Atm) comprised from 6±0,1 to 8±0,1, preferably comprised from 6,5±0,1 to 7,5±0,1, even more preferably from 6.7±0,1 to 7,3±0,1.

G10. The composition for use according to any one of G1-G9, wherein said method comprises an administration of said composition through the ocular topical route, preferably on the ocular adnexa or on the eyeball, more preferably on the eyelid, conjunctiva, cornea and/or vitreous, even more preferably on the cornea (with the eye open) or on the eyelid (with the eye closed).

G11. The composition for use according to any one of G1-G10, wherein said disorder or said ailment or said disease is an eye inflammation selected from the group comprising, or alternatively, consisting of allergic conjunctivitis, allergic keratitis, keratitis, dry eye syndrome, allergy eye redness, and swelling from ocular phlogosis.

G12. A method for the curative treatment of an inflammation of the ocular adnexa or inflammation of the eyeball in a subject in need, wherein said treatment method comprises administering the composition according to any one of G1-G11, to said subject.

1. A composition comprising a mixture comprising, or alternatively, consisting of:
(i) a budesonide, or a salt thereof, or mixtures thereof, in form of micronized powder, wherein said micronized powder comprises powder particles having an average size distribution comprised from 0,05 μm to 20 μm;
(ii) at least one suspending/thickening agent selected from the group comprising or, alternatively, consisting of: carboxymethylcellulose (CMC), sodium carboxymethylcellulose (Na-CMC) and refined carboxymethylcelluloses, or mixtures thereof, wherein said composition comprises only the water-soluble fractions of CMC, Na-CMC and/or refined carboxymethylcelluloses alone, and optionally (a.2) or more physiologically and/or pharmacologically acceptable excipient,
wherein said composition is for use in a method for the curative treatment of an inflammation of the ocular adnexa or an inflammation of the eyeball in a subject in need.

2. Composition for use according to claim 1, wherein an amount of said (i) budesonide, or salt thereof, or mixtures thereof is comprised from 0,005% (w/v) to 3% (w/v) by weight with respect to a total volume of said composition, preferably comprised from 0,01% (w/v) to 2% (w/v), even more preferably comprised from 0.05% (w/v) to 1,5% (w/v), for example 0.1% (w/v), 0,2% (w/v) or 0.5% (w/v).

3. The composition for use according to claim 1, wherein said refined carboxymethylcelluloses have a degree of substitution (DS), defined as the average number of hydroxyl groups substituted with anhydroglucose units in said refined CMCs, comprised from 0,65 to 0,90 or comprised from 0,80 to 0,95, preferably comprised from 0,80 to 0,95.

4. The composition for use according to claim 1, wherein said one or more physiologically and/or pharmacologically acceptable excipients is selected from the group comprising or, alternatively, consisting of: A preservative, preferably potassium sorbate and/or benzalkonium chloride, a surfactant, preferably polyoxyethylene (20) sorbitan monooleate, a chelating agent, preferably ethylenediaminetetraacetic acid (EDTA), or a salt thereof, or mixtures thereof, an isotonic agent, preferably mannitol or sodium chloride, a carrier, preferably an aqueous carrier, more preferably water, or mixtures thereof.

5. Composition for use according to claim 1, comprising:
(i) a budesonide, or a salt thereof, or mixtures thereof;
(ii) cellulose, carboxymethylcellulose (CMC), sodium carboxymethylcellulose (Na-CMC), and/or refined carboxymethylcelluloses, preferably in cross-linked form/s, or mixtures thereof;
(iii.a) potassium sorbate;
(iii.b) polyoxyethylene (20) sorbitan monooleate (Polysorbate 80);
(iii.c) ethylenediaminetetraacetic acid (EDTA), or a salt thereof, or mixtures thereof;
(iii.d) mannitol or sodium chloride;
(iii.e) benzalkonium chloride;
(iii.f) water, preferably purified water or water for injections;
preferably, said composition being devoid of opaque ingredients, i.e., ingredients additional to said (i) budesonide, or salt thereof, or mixtures thereof suspended in said composition.

6. The composition for use according to claim 5, comprising (amounts expressed as weight with respect to the total volume of said composition; w/v):
(i) a budesonide, or a salt thereof, or mixtures thereof comprised from 0,005% (w/v) to 3% (w/v), preferably comprised from 0,01% (w/v) to 2% (w/v), even more preferably comprised from 0.05% (w/v) to 1,5% (w/v), for example 0.1% (w/v), 0,2% (w/v) or 0.5% (w/v);
(ii) cellulose, carboxymethylcellulose (CMC), sodium carboxymethylcellulose (Na-CMC), refined carboxymethylcelluloses preferably in cross-linked form/s, or mixtures thereof comprised from 0,005% (w/v) to 3% (w/v), preferably comprised from 0,01% (w/v) to 2% (w/v), more preferably comprised from 0,01% (w/v) to 2% (w/v) even more preferably comprised from 0.05% (w/v) to 1,5% (w/v), for example 0,160% (w/v);
(iii.a) potassium sorbate comprised from 0,005% (w/v) to 3% (w/v), preferably comprised from 0,01% (w/v) to 2% (w/v), more preferably comprised from 0.05% (w/v) to 1,5% (w/v), for example 0,100% (w/v);
(iii.b) polyoxyethylene (20) sorbitan monooleate (Polysorbate 80) comprised from 0.001% (w/v) to 2% (w/v), preferably comprised from 0,005% (w/v) to 1% (w/v), even more preferably comprised from 0,01% (w/v) to 1,5% (w/v), for example 0,030% (w/v);
(iii.c) ethylenediaminetetraacetic acid (EDTA), or a salt thereof, or mixtures thereof comprised from 0,005% (w/v) to 3% (w/v), preferably comprised from 0,01% (w/v) to 2% (w/v), more preferably comprised from 0.05% (w/v) to 1,5% (w/v), for example 0,100% (w/v);
(iii.d) mannitol or sodium chloride comprised from 0,01% (w/v) to 10% (w/v), preferably comprised from 0,1% (w/v) to 7% (w/v), more preferably comprised from 0.5% (w/v) to 5% (w/v), even more preferably comprised from 0,8% (w/v) to 3% (w/v), for example 1,800% (w/v);
(iii.e) benzalkonium chloride comprised from 0,005% (w/v) to 3% (w/v), preferably comprised from 0,01% (w/v) to 2% (w/v), more preferably comprised from 0.05% (w/v) to 1,5% (w/v), for example 0,100% (w/v);
(iii. f) water, preferably purified water or water for injections, up to a volume of 10 ml of said composition.

7. The composition for use according to claim 1, wherein said micronized powder comprises powder particles having an average size distribution comprised from 0,1 μm to 10 μm, preferably comprised from 0,2 μm to 5 μm, more preferably comprised from 0.25 μm to 3,75 μm.

8. The composition for use according to claim 1, wherein said micronized powder comprises powder particles having an average size distribution comprised from 0,40 μm to 3,0 μm.

9. The composition for use according to claim 1, wherein said composition is a suspension in water, preferably wherein said suspension has a pH value (at 20° C. and 1 Atm) comprised from 6±0,1 to 8±0,1, preferably comprised from 6,5±0,1 to 7,5±0,1, even more preferably from 6.7±0,1 to 7,3±0,1.

10. The composition for use according to claim 1, wherein said method comprises an administration of said composition through the ocular topical route, preferably on the ocular adnexa or on the eyeball, more preferably on the eyelid, conjunctiva, cornea and/or vitreous, even more preferably on the cornea (with eye open) or on the eyelid (with the eye closed).

11. The composition for use according to claim 1, wherein said disorder or said ailment or said disease is an eye inflammation selected from the group comprising, or alternatively, consisting of allergic conjunctivitis, allergic keratitis, keratitis, dry eye syndrome, allergy eye redness, and swelling from ocular phlogosis.

12. A method for the curative treatment of an inflammation of the ocular adnexa or inflammation of the eyeball in a subject in need, wherein said treatment method comprises administering a composition comprising a mixture comprising, or alternatively, consisting of:
(i) a budesonide, or a salt thereof, or mixtures thereof, in form of micronized powder, wherein said micronized powder comprises powder particles having an average size distribution comprised from 0.05 μm to 20 μm;
(ii) at least one suspending/thickening agent selected from the group comprising or, alternatively, consisting of: carboxymethylcellulose (CMC), sodium carboxymethylcellulose (Na-CMC) and refined carboxymethylcelluloses, or mixtures thereof, wherein said composition comprises only the water-soluble fractions of CMC, Na-CMC and/or refined carboxymethylcelluloses alone, and optionally
(a.2) or more physiologically and/or pharmacologically acceptable excipient to said subject.

13. The method of claim 12, wherein said (i) budesonide, or salt thereof, or mixtures thereof is present in the composition from 0,005% (w/v) to 3% (w/v) by weight with respect to a total volume of said composition, preferably comprised from 0,01% (w/v) to 2% (w/v), even more preferably comprised from 0.05% (w/v) to 1,5% (w/v), for example 0.1% (w/v), 0,2% (w/v) or 0.5% (w/v).

14. The method of claim 12, wherein said refined carboxymethylcelluloses have a degree of substitution (DS), defined as the average number of hydroxyl groups substituted with anhydroglucose units in said refined CMCs, comprised from 0,65 to 0,90 or comprised from 0,80 to 0,95, preferably comprised from 0,80 to 0,95.

15. The method of claim 12, wherein said one or more physiologically and/or pharmacologically acceptable excipients is selected from the group comprising or, alternatively, consisting of: A preservative, preferably potassium sorbate and/or benzalkonium chloride, a surfactant, preferably polyoxyethylene (20) sorbitan monooleate, a chelating agent, preferably ethylenediaminetetraacetic acid (EDTA), or a salt thereof, or mixtures thereof, an isotonic agent, preferably mannitol or sodium chloride, a carrier, preferably an aqueous carrier, more preferably water, or mixtures thereof.

16. The method of claim 12, wherein the composition comprises:
(i) a budesonide, or a salt thereof, or mixtures thereof;
(ii) cellulose, carboxymethylcellulose (CMC), sodium carboxymethylcellulose (Na-CMC), and/or refined carboxymethylcelluloses, preferably in cross-linked form/s, or mixtures thereof;
(iii.a) potassium sorbate;
(iii.b) polyoxyethylene (20) sorbitan monooleate (Polysorbate 80);
(iii.c) ethylenediaminetetraacetic acid (EDTA), or a salt thereof, or mixtures thereof;
(iii.d) mannitol or sodium chloride;
(iii.e) benzalkonium chloride;
(iii.f) water, preferably purified water or water for injections;
preferably, said composition being devoid of opaque ingredients, i.e., ingredients additional to said (i) budesonide, or salt thereof, or mixtures thereof suspended in said composition.

17. The method of claim 12, wherein the composition comprises (amounts expressed as weight with respect to the total volume of said composition; w/v):
(i) a budesonide, or a salt thereof, or mixtures thereof comprised from 0,005% (w/v) to 3% (w/v), preferably comprised from 0,01% (w/v) to 2% (w/v), even more preferably comprised from 0.05% (w/v) to 1,5% (w/v), for example 0.1% (w/v), 0,2% (w/v) or 0.5% (w/v);
(ii) cellulose, carboxymethylcellulose (CMC), sodium carboxymethylcellulose (Na-CMC), refined carboxymethylcelluloses preferably in cross-linked form/s, or mixtures thereof comprised from 0,005% (w/v) to 3% (w/v), preferably comprised from 0,01% (w/v) to 2% (w/v), more preferably comprised from 0,01% (w/v) to 2% (w/v) even more preferably comprised from 0.05% (w/v) to 1,5% (w/v), for example 0,160% (w/v);
(iii.a) potassium sorbate comprised from 0,005% (w/v) to 3% (w/v), preferably comprised from 0,01% (w/v) to 2% (w/v), more preferably comprised from 0.05% (w/v) to 1,5% (w/v), for example 0,100% (w/v);
(iii.b) polyoxyethylene (20) sorbitan monooleate (Polysorbate 80) comprised from 0.001% (w/v) to 2% (w/v), preferably comprised from 0,005% (w/v) to 1% (w/v), even more preferably comprised from 0,01% (w/v) to 1,5% (w/v), for example 0,030% (w/v);
(iii.c) ethylenediaminetetraacetic acid (EDTA), or a salt thereof, or mixtures thereof comprised from 0,005% (w/v) to 3% (w/v), preferably comprised from 0,01% (w/v) to 2% (w/v), more preferably comprised from 0.05% (w/v) to 1,5% (w/v), for example 0,100% (w/v);
(iii.d) mannitol or sodium chloride comprised from 0,01% (w/v) to 10% (w/v), preferably comprised from 0,1% (w/v) to 7% (w/v), more preferably comprised from 0.5% (w/v) to 5% (w/v), even more preferably comprised from 0,8% (w/v) to 3% (w/v), for example 1,800% (w/v);
(iii.e) benzalkonium chloride comprised from 0,005% (w/v) to 3% (w/v), preferably comprised from 0,01% (w/v) to 2% (w/v), more preferably comprised from 0.05% (w/v) to 1,5% (w/v), for example 0,100% (w/v);
(iii. f) water, preferably purified water or water for injections, up to a volume of 10 ml of said composition.

18. The method of claim 12, wherein said micronized powder comprises powder particles having an average size distribution comprised from 0,1 μm to 10 μm, preferably comprised from 0,2 μm to 5 μm, more preferably comprised from 0.25 μm to 3,75 μm.

19. The method of claim 12, wherein said method comprises an administration of said composition through the ocular topical route, preferably on the ocular adnexa or on the eyeball, more preferably on the eyelid, conjunctiva, cornea and/or vitreous, even more preferably on the cornea (with eye open) or on the eyelid (with the eye closed).

20. The method of claim 12, wherein said disorder or said ailment or said disease is an eye inflammation selected from the group comprising, or alternatively, consisting of allergic conjunctivitis, allergic keratitis, keratitis, dry eye syndrome, allergy eye redness, and swelling from ocular phlogosis.
